(19) United States
(12) Patent Application Publication
(10) Pub. No.: US 2007/0038034 A1
(43) Pub. Date: Feb. 15, 2007

(54) Systems and methods for performing percutaneous surgery

(76) Inventor: Thomas M. Sweeney II, Sarasota, FL (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/505,211

(22) Filed: Aug. 15, 2006

Related U.S. Application Data
(60) Provisional application No. 60/708,731, filed on Aug. 15, 2005.

(51) Int. Cl. A61B 1/32

(52) U.S. Cl. 600/219

(57) ABSTRACT

Percutaneous tooling and methods are used to perform a minimally invasive surgery. The tool includes an elongated driver and a number of blade members. The blades are movable from a retracted position to an expanded position. The percutaneous method includes inserting a wire selectively through the tissue surrounding a surgical site. The wire is then used as a guide for other tools, such as a drill and/or screw tap to prepare an opening in the bone. The other tools may be selectively guided over the wire and through a passageway formed within the tissue by the wire. The passageway may be formed through interstices present in the tissue, which at least minimizes damage and trauma to the tissue in vicinity of the surgical site.

### RELATED APPLICATIONS

This application claims the benefit under 35 U.S.C. §119(e) of U.S. Provisional Patent Application No. 60/708,731, filed Aug. 15, 2005 titled “Minimally Invasive Surgical Retractor,” which application is incorporated herein by reference in its entirety.

### FIELD

The present system and method relate to devices and methods for performing percutaneous surgeries, and more particularly, to percutaneous surgical tooling and methods for minimizing tissue damage within and near a surgical site.

### BACKGROUND

Traditionally, the surgical exposure employed to perform spinal surgery inflicts significant and long lasting damage to the surrounding soft tissues. Surgical exposure, commonly referred to as an ‘open’ procedure, relies on retraction of muscles to open a channel to the underlying bony structures. Surgical retractors are often used to provide the operating channel. Common surgical retractors as used in the art today include rakes, forks, and different sized and shaped hooks. Normally, the hooks are constructed of a stainless steel or latex-free silicon so that they may be used in the sterile environment of the surgery. While such retractors as rakes or hooks are useful for certain types of injury, extreme care must be used to ensure that the retractor does not cause additional damage to the wound. In addition, use of the surgical retractor may require two, three, or more additional assistants to the physician, with appropriate training, in order to hold the retractor in the correct position so that the site of the surgery is more easily accessible to the physician. Other types of surgical retractors are inserted into the surgical site and then one or more arms are spread in order to open the insertion site for further access by the physician. These retractors are generally bulky, require substantial training and skill to operate, and user error may increase the difficulty and the time for the surgery. Traditional retraction using the above-mentioned retractors is recognized to cut-off circulation to the muscles and often results in post-operative pain and long-term degradation of muscle function.

Recently, minimally invasive techniques have been developed to reduce the intra-operative damage and reduce the post-operative recovery time. In minimally invasive surgery (MIS), a desired site is accessed through portals rather than through a significant incision. Various types of access portals have been developed for use in MIS. Many of the existing MIS access portals, such as those described in U.S. Pat. Nos. 4,573,488 and 5,395,317 issued to Kambin, can only be used for a specific procedure. Other prior art portals, such as that described in U.S. Pat. No. 5,439,464 issued to Shapiro, require multiple portals into the patient, adding complexity to the portal placement as well as obstructing the operating space.

### SUMMARY

According to one exemplary embodiment, the tools and methods described herein provide a variety of ways to minimize the trauma and damage that may occur to the tissue in the vicinity of a surgical site. In one example, reducing the trauma and damage to the paraspinous tissue during a spinal surgery allows the patient to strengthen their back muscles quicker and also recover faster.

In one exemplary embodiment, a tool includes an elongated driver having an expander and a plurality of blades each having a proximal portion and a distal portion. According to this exemplary embodiment, the distal portions of the plurality of blades are positioned around and proximate to the expander. The plurality of blades is moveable from a retracted position to an expanded position. When in the retracted position, the distal portions of the plurality of blades are located a first distance from the driver. However, when in the expanded position, the distal portions of the plurality of blades are located further from the driver than when in the retracted position.

In another exemplary embodiment, a method for percutaneously preparing a vertebrae to receive a screw includes first inserting a wire through at least several layers of human tissue including paraspinous tissue, guiding the wire through interstices present in the paraspinous tissue, contacting at least a portion of the vertebrae with the wire, guiding a tool over the wire and through the interstices, forming a passage in the vertebrae, and forming internal screw threads in the passage.

In yet another exemplary embodiment, a percutaneous method for inserting a screw into a bone includes inserting a driver coupled to the screw through tissue present around the bone, turning the screw into the bone with the driver, and inserting a cannula through the tissue.

The foregoing is a summary and thus contains, by necessity, simplifications, generalizations and omissions of detail; consequently, those skilled in the art will appreciate that the summary is illustrative only and is not intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is a cross-sectional view of a surgical site, according to one exemplary embodiment.

FIG. 2 is a side elevation view of a surgical tool including a number of blades in a retracted position, according to one exemplary embodiment.

FIG. 3 is a top, front, isometric, partial view of the blades of the surgical tool of FIG. 2 shown in an expanded position, according to one exemplary embodiment.

FIG. 4 is a side elevation view of the surgical tool of FIG. 2 in an expanded position, according to one exemplary embodiment.

FIG. 5 is a cross-sectional view of the surgical site of FIG. 1 with a wire inserted into a vertebral body, according to one exemplary embodiment.

FIG. 6 is a cross-sectional view of the surgical site of FIG. 1 with a drill placed over the wire of FIG. 5, according to one exemplary embodiment.

FIG. 7 is a cross-sectional view of the surgical site of FIG. 1 with a screw tap placed over the wire of FIG. 5, according to one exemplary embodiment.

FIG. 8 is a cross-sectional view of the tool of FIG. 2 with a sleeve located over the blades and the tool positioned in the surgical site of FIG. 1, according to one exemplary embodiment.

FIG. 9 is a cross-sectional view of the tool of FIG. 8 in the retracted position and showing the sleeve being removed, according to one exemplary embodiment.

FIG. 10 is a cross-sectional view of the tool of FIG. 8, without the sleeve, in the expanded position, according to one exemplary embodiment.

FIGS. 11-13 are side elevation views of a minimally invasive surgical (MIS) retractor according to three exemplary embodiments.

Throughout the drawings, identical reference numbers designate similar but not necessarily identical elements. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

### DETAILED DESCRIPTION

In the following description, various details are set forth in order to provide a thorough understanding of a variety of embodiments of the present tools, assemblies, systems, and methods. However, one skilled in the relevant art will recognize that the tools, assemblies, systems, and methods described herein may be practiced without one or more of these specific details, or with other methods, components, materials, etc. In other instances, well-known structures associated with surgical tooling have not been shown or described in detail to avoid unnecessarily obscuring descriptions of the embodiments of the present assemblies, devices and systems.

Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, the particular features, structures, or characteristics may be combined in any suitable manner in one or more embodiments.

Unless the context requires otherwise, throughout the specification and claims which follow, the word “comprise” and variations thereof, such as, “comprises” and “comprising” are to be construed in an open, inclusive sense; that is as “including, but not limited to.”

Additionally, the headings provided herein are for convenience only and do not interpret the scope or meaning of the claimed invention.

While the present system and method may be practiced by or incorporated into any number of systems, the present system and method will be described herein, for ease of explanation only, in the context of percutaneous tooling and methods for use in orthopedic spinal surgery; providing a channel to the underlying bony structures of the spine while minimizing trauma to the overlying tissues. According to aspects of the present exemplary system and method, the driver and expander assembly is able to minimize the need for muscle retraction. The features and advantages of the exemplary systems and methods will be set forth in the description which follows, and in part will be apparent from the description.

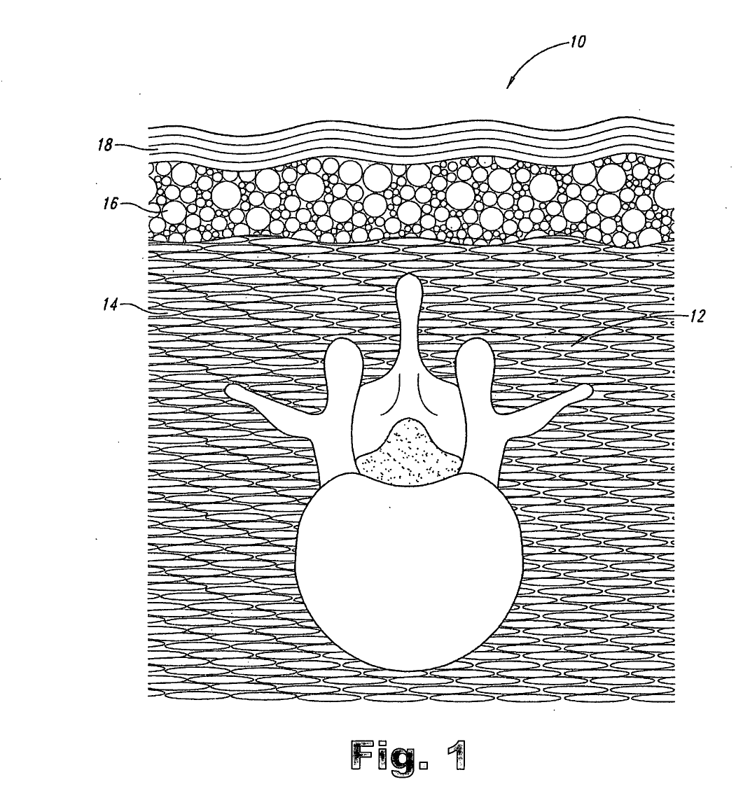

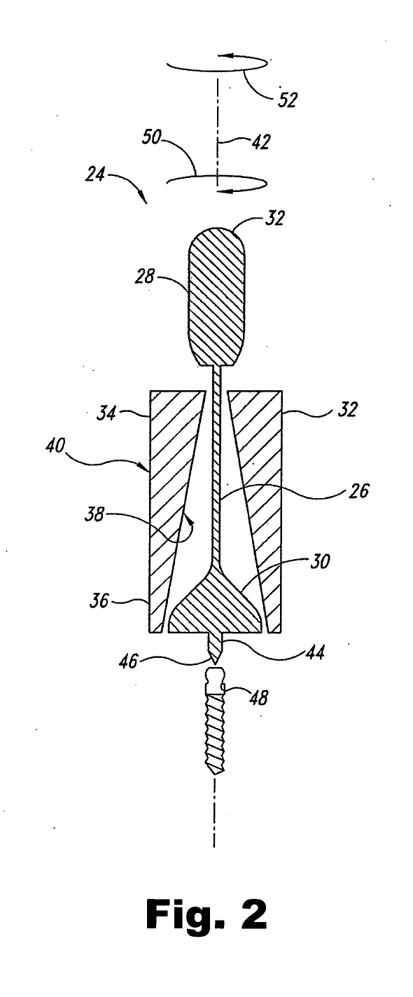

FIG. 1 generally illustrates a surgical site (10) that may be accessed by the present exemplary system and method. As illustrated in FIG. 1, the surgical site (10) includes a vertebral body (12) located in paraspinous tissue (14), including the multifidus muscle. According to the exemplary surgical site (10) shown in FIG. 1, a fatty layer (16) is located between the paraspinous tissue (14) and a dermal layer (18). The exemplary embodiments of the tooling and the methods described below are configured to facilitate percutaneous access to the surgical site (10) during a surgical procedure. As used herein, and in the appended claims, the term “percutaneous” is meant to be interpreted broadly as including any medical procedure where access to inner organs, bone structure, or other tissue is performed by a needle puncture of the skin, rather than by using an open approach where inner organs or tissue are exposed via a scalpel incision. Particular details of the present exemplary system and method will be provided below with reference to FIGS. 2-13.

Percutaneous Surgical Tooling

FIG. 2 illustrates a percutaneous tool (24) having an elongated body driver (26), a handle (28), and an expander (30), according to one exemplary embodiment. As shown, the handle (28) is positioned on the upper portion of the tool (24) while the expander (30) is positioned on the lower portion according to the illustrated embodiment.

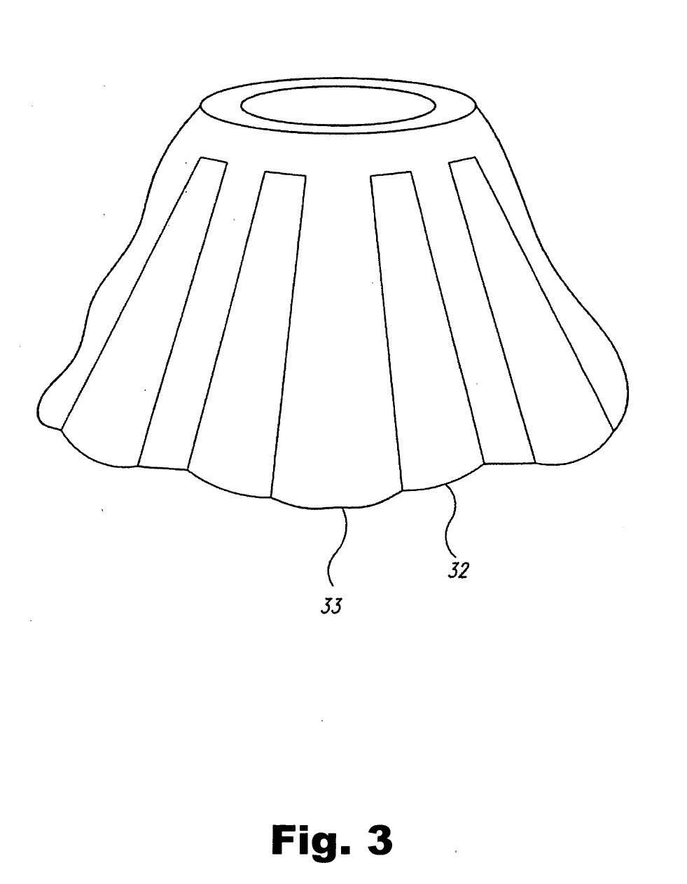

Additionally, a number of blade members (32) are positioned approximately around the driver (26). According to the present exemplary embodiment, each blade member (32) includes one or more flexible membranes (33; FIG. 3), a proximal portion (34), a distal portion (36), and a cam surface (38). As will be described in detail below, the cam surface (38) operates in conjunction with the expander (30) to translate the blade members (32) from a retracted position to an expanded position, best seen in FIG. 3, and/or any number of intermediate positions.

According to one exemplary embodiment illustrated in FIG. 3, a number of flexible membranes (33) are disposed between and coupled to each of the blade members (32) such that the combination of blade members and flexible membranes form a substantially continuous member. According to this exemplary embodiment, the flexible membranes (33) operate to support, urge, and maintain the paraspinous tissue (14), the fatty layer (16), and/or the dermal layer (18) away from the surgical site (10) when the blade members (32) are in an expanded position. The number of blade members (32) and membranes (33) positioned around the driver (26) may vary depending on the type of procedure that is to be performed, the density of the tissue that is to be moved away from the surgical site (10), the strength of the flexible membranes (33), and/or other factors.

Referring back to FIG. 2, when the tool (24) is in a retracted position, outer circumferences (40) of the blade members (32) are generally, cylindrically aligned with a longitudinal axis (42) of the driver (26), according to one exemplary embodiment. Particularly, when in the retracted position, the cam surfaces (38) on the proximal portions (34) of the blade members (32) are closely located to, but not necessarily in contact with, the upper portion of the driver (26). In addition, the cam surfaces (38) on the distal portions (36) of the blade members (32) are located proximate, and may be in contact with, the expander (30). In one exemplary embodiment of the retracted position of the tool (24), the cam surfaces (38) on the distal portions (36) of the blade members (32) are located approximately 0.300-0.500 inches from the driver (26). This numerical range may be adequate for certain types of surgeries, however it is understood that this range may be expanded if the tool (24) is customized for a different procedure or used for a different purpose.

In addition to the above structures, the driver (26), according to one exemplary embodiment, includes the expander (30) having a campana shaped profile and a coupling member (44) protruding there from. According to one exemplary embodiment, the coupling member (44) is configured to frictionally couple and at least temporarily retain a screw (48), such as a pedicle screw that is to be inserted into the vertebral body (12). In one exemplary embodiment, the coupling member (44) includes a number of serrations (46) configured to engage and retain the screw (48) when the driver (26) is rotated in a first direction (50). After the screw (48) is placed in a bone, or the vertebral body (12) for instance, the driver (26) can be turned in a second direction (52) to release the serrations (46) of the coupling member (44) from the screw (48). One exemplary type of pedicle screw (48) that may be used with the present exemplary system and method is described in detail in a U.S. Patent Application, filed on Jul. 28, 2006, entitled “Thread on a Bone Screw,” having Express Mail No. EV 895433933 US and corresponding to Attorney Docket No. 40359-0064, which application is incorporated herein by reference in its entirety. Additionally, the present exemplary systems and methods are in no way limited to use with a pedicle screw. Rather, the present systems and methods may be used with any number of orthopedic fasteners or implants.

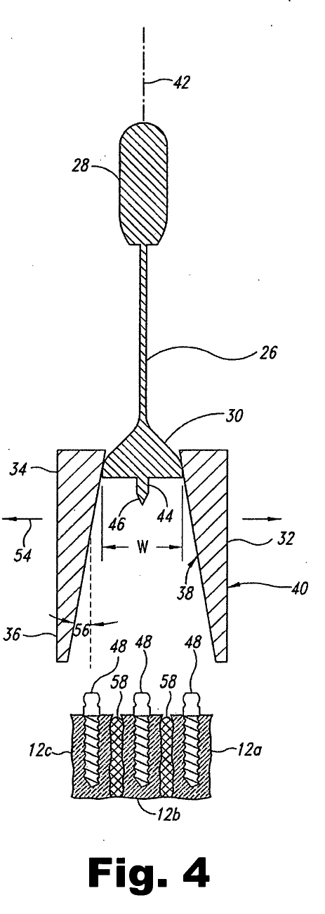

Continuing with the figures, FIG. 4 shows the present exemplary tool (24) with the blade members (32) in the expanded position, according to one exemplary embodiment. As illustrated, when the driver (26) is released from the screw (48) or other fastener, the driver is pulled away from the surgical site (10), which causes the expander (30) to contact the cam surface (38) and move the blade members (32) apart from one another and away from the longitudinal axis (42) as indicated by the arrows (54). It is understood that the inclination angle (56) of the cam surface (38) formed on the inner surface of the blade members (32) and/or the width of the expander (30) can be varied or customized to achieve a variety of retracted and expanded positions. In one exemplary embodiment, the width “W of the expander (30) is in the range of about 0.600-1.600 inches. Additionally or alternatively, the cam surface 38 can be curved to achieve a custom, kinematic expansion of the blade members (32).

It is also understood that the distal portions (36) of the blade members (32) may be expanded an amount that provides a surgeon access to the complete surgical site (10) within the region formed by the expanded blade members (32). In the illustrated and exemplary embodiment, the blade members (32) are expanded over the vertebral bodies (12a, 12b, 12c), which have intervertebral disks (58) located therebetween. Thus, the surgeon can access a number of adjacent screws (48) that may have been previously secured into the vertebral bodies (12a, 12b, 12c), for example. It is understood and appreciated that the surgeon controls the amount of expansion of the blade members (32) by selective manipulation of the driver (26). Further details of the operation of the present exemplary percutaneous tool (24) will be provided below.

Percutaneous Surgical Method

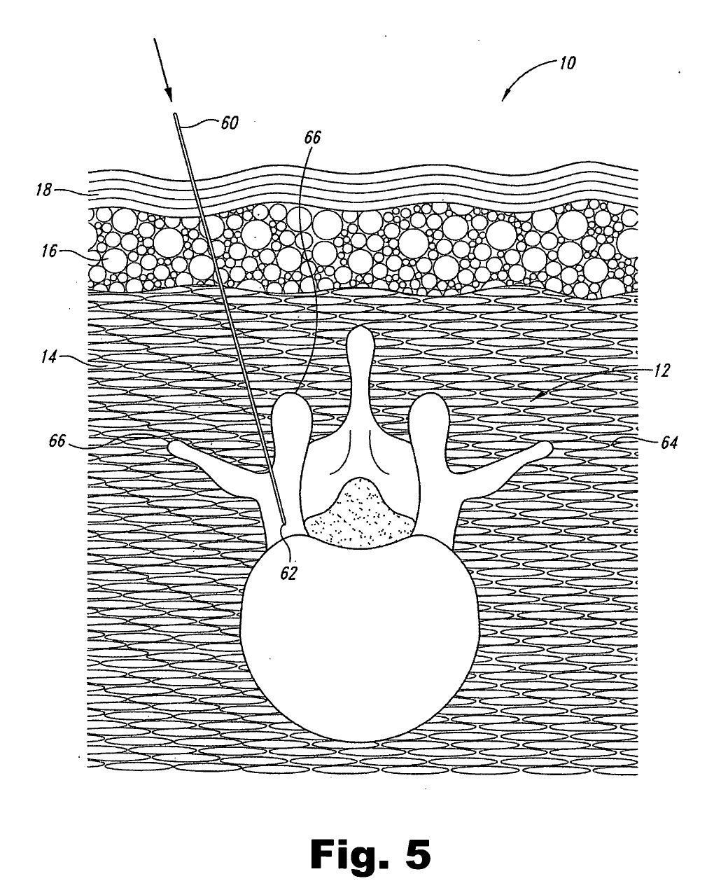

FIGS. 5-8 generally illustrate a method of accessing the vertebral body (12) through the various layers of tissue (14, 16, 18), for example. According to one exemplary embodiment, the present method is a minimally invasive method that can be used during a spinal surgery to minimize trauma and damage to the paraspinous tissue (14) surrounding the vertebral body (12).

FIG. 5 illustrates the surgical site (10) of FIG. 1 surrounded by the paraspinous tissue (14), according to one exemplary embodiment. As illustrated in FIG. 1, a wire (60), having a tip (62), is inserted into a portion of the surgical site (10). In one exemplary embodiment, the wire (60) is a k-wire and forms a small, needle-sized opening in the tissue layers (16,18). The wire (60) is further inserted through interstices (64), i.e., separations that occur between the natural pathways of the muscle fibers and/or striations, in the paraspinous tissue (14), and more particularly in the multifidus muscle. Finally, the wire (60) is the inserted into contact with and may even be urged into the cancellous bone of the vertebral body (12) to a desired depth. In the illustrated embodiment, the wire (60) is inserted between adjacent pedicles (66) extending from the vertebral body (12).

The insertion of the wire (60) establishes a small channel through the tissue layers (14,16) and further through the paraspinous tissue (14). One advantage of finding and then guiding the wire (60) through the interstices (64) is to avoid puncturing, cutting, or otherwise damaging the paraspinous tissue (14), in particular the multifidus muscle. In one exemplary embodiment, the wire (60) is guided, as described above, with the assistance of an x-ray imager, a fluoroscopic imager, some other type of two and/or three-dimensional imager, and/or some combination of the above.

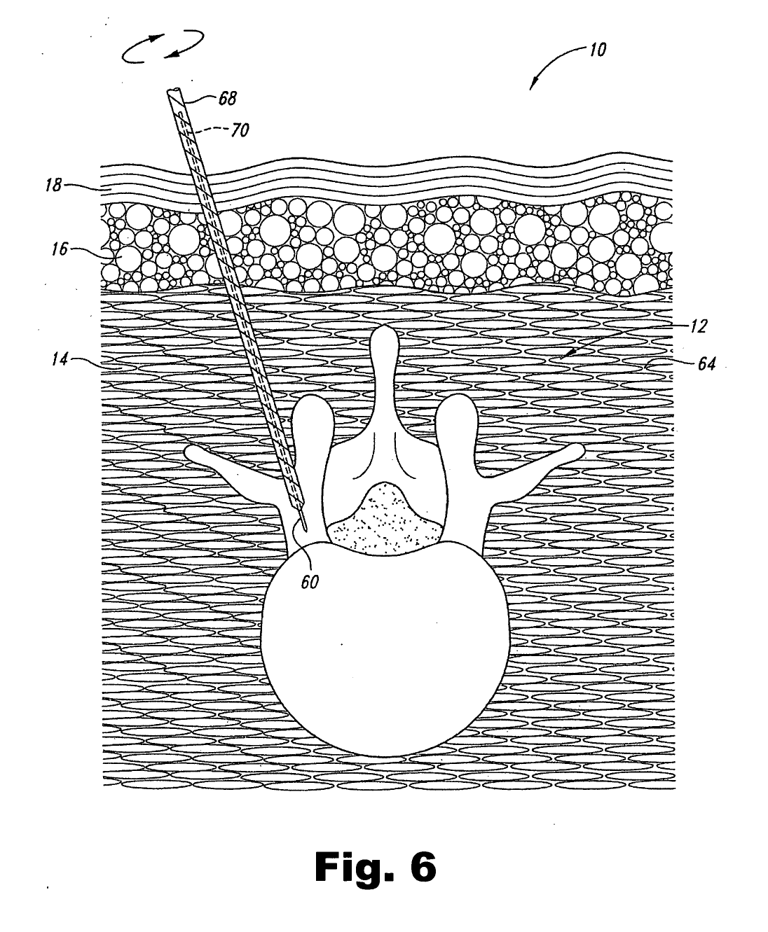

Once the wire (60) is placed, a drill may be passed over the wire. FIG. 6 shows a drill (68) having a central passage (70) configured to receive the wire (60). Once inserted over the wire (60), the drill (68), using the wire as a guide, is directed through the interstices (64) toward the vertebral body (12). The drill (68) can be operated to drill an opening in the vertebral body (12). In one exemplary embodiment, the diameter of the drill (68)I is sized to create an opening for receiving a pedicle screw (48), such as the pedicle screw described in detail above.

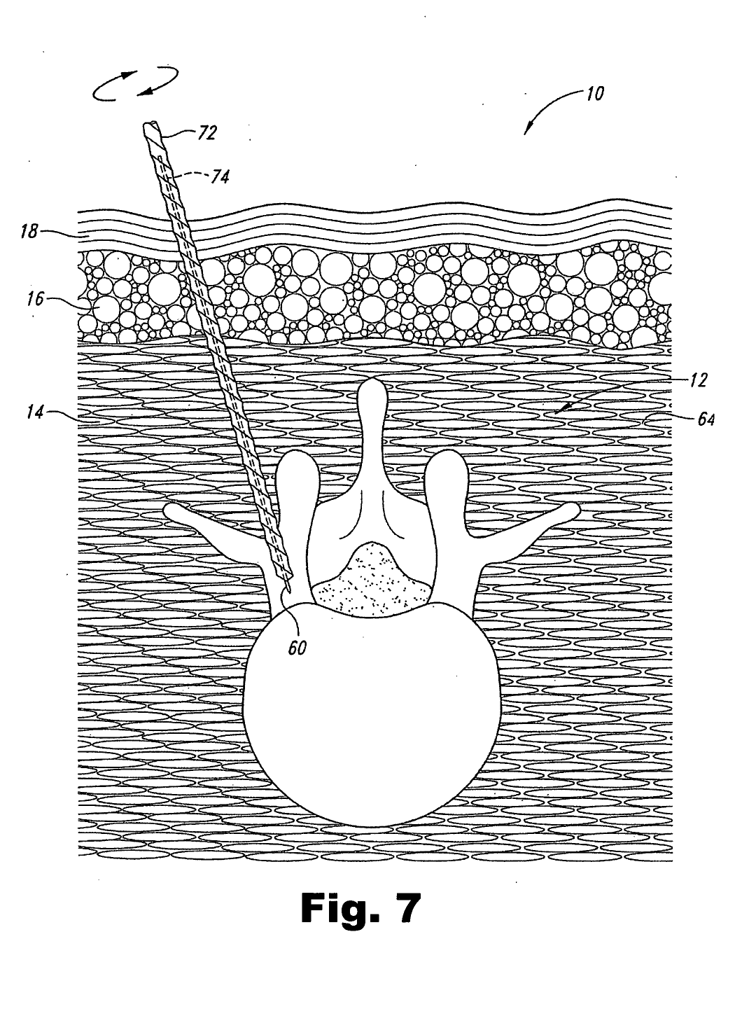

With the desired opening drilled in the vertebral body (12), internal threads may be formed in the vertebral body, according to one exemplary embodiment. FIG. 7 illustrates a screw tap (72) having a central passage (74) configured to receive the wire (60). According to one exemplary embodiment, the tap (72), using the wire (60) as a guide, is also directed through the interstices (64) toward the vertebral body (12). The tap (60) can then be operated to produce internal screw threads in the opening made by the drill (68). Alternatively, a self-tapping screw (48) may be used to simultaneously form the desired orifice and threads.

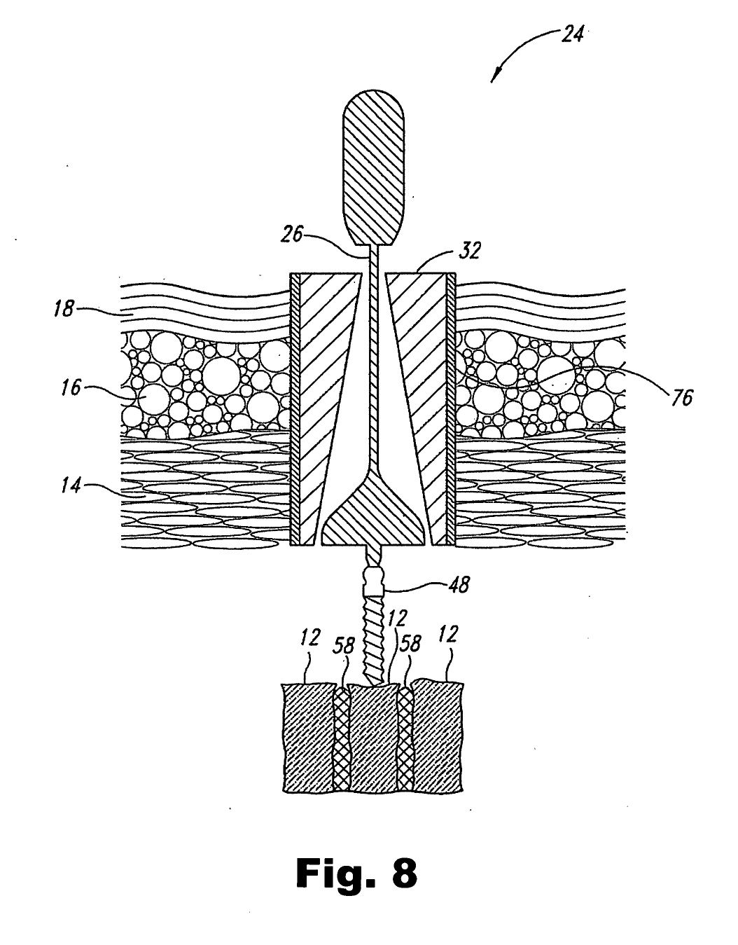

According to one exemplary embodiment, once the desired orifice is formed and threaded, as described above, the driver and blade assembly may be inserted through the orifice and guided down the wire (60). FIG. 8 illustrates the driver (26) coupled to the screw (48) and the blade members (32) being inserted through the tissue layers (14,16,18), according to one exemplary embodiment. As shown, the blade members (32) are in the retracted position when inserted and are coupled to the driver (26) via a removable sleeve (76) that closely fits over the blade members (32) and flexible membranes (33) (not shown) according to one exemplary embodiment. While one exemplary method for coupling the blade members (32) to the driver (26) is illustrated in FIG. 8, it is understood that the blade members and the flexible membranes (33) may be coupled to the driver (26) by any other type of coupling means.

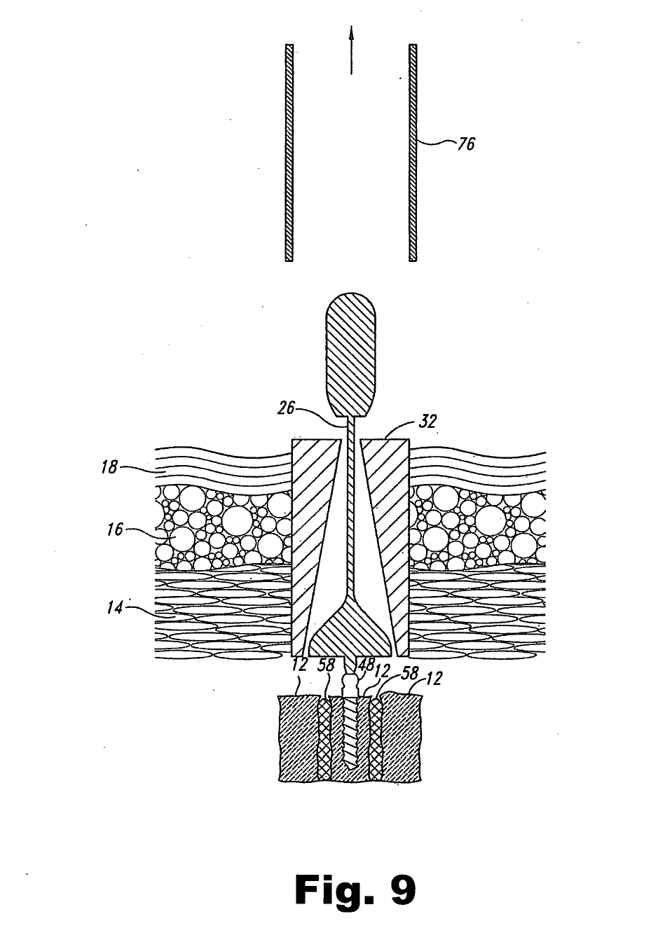

After the driver (26) and blade members (32) have been inserted to a desired position through the tissue layers (14,16, 18), the sleeve (76) may be removed to allow for further operations. FIG. 9 illustrates the sleeve (76) being slid upward and off of the blade members (32), according to one exemplary embodiment. The sleeve (76) may be slid off of the blade members (32) either before or after the screw (48) is inserted into the vertebral body (12). With the sleeve (76) removed, the blade members (32) are free to expand when acted upon by the driver (26), as mentioned previously.

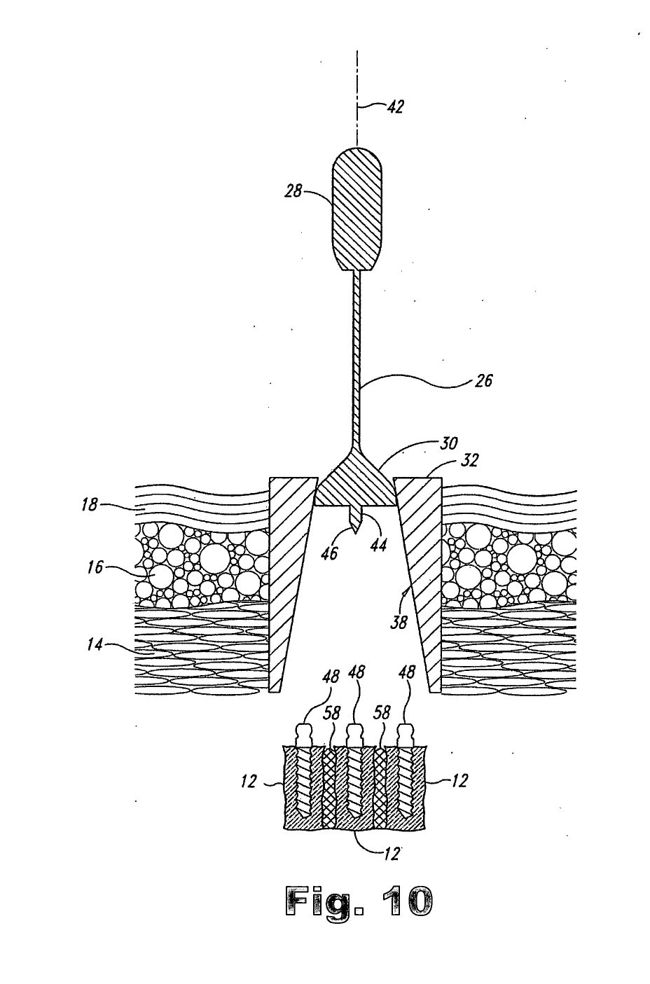

Specifically, FIG. 10 shows the expander (30) as it has moved upward and through the blade members (32) to expand the blade members in the tissue (14, 16,18), according to one exemplary embodiment. As illustrated, once the expander (30) has actuated the blade members (32), the blade members are wide enough apart to permit access to several intervertebral disks (58) and the associated vertebral bodies (12). Once the blade members (32) are in place, the driver (26) can be completely removed. The channel formed by the blade members (32) and membranes (33) provides a clean and clear access site over the vertebral bodies (12), or some other desired site. As the blade members (32) are expanded, the blade members (32) and membranes (33) gently urge the tissue (14) away from the vertebral body (12).

Once the blade members (32) are in their expanded position, they allow the surgeon to work in a variety of locations within the surgical site (10), which may comprise a number of intervertebral disks (58) located between portions of the vertebral body (12). By way of example, the surgeon may determine that a compressed disk (58) needs to be repaired, which may involve separating and then fusing adjacent portions of the vertebral body (12) together using a pedicle screw system. One type of pedicle screw system and the installation thereof is described in detail in U.S. Provisional Patent Application No. 60/665,032, filed on Mar. 23, 2005, which application is incorporated herein by reference in its entirety.

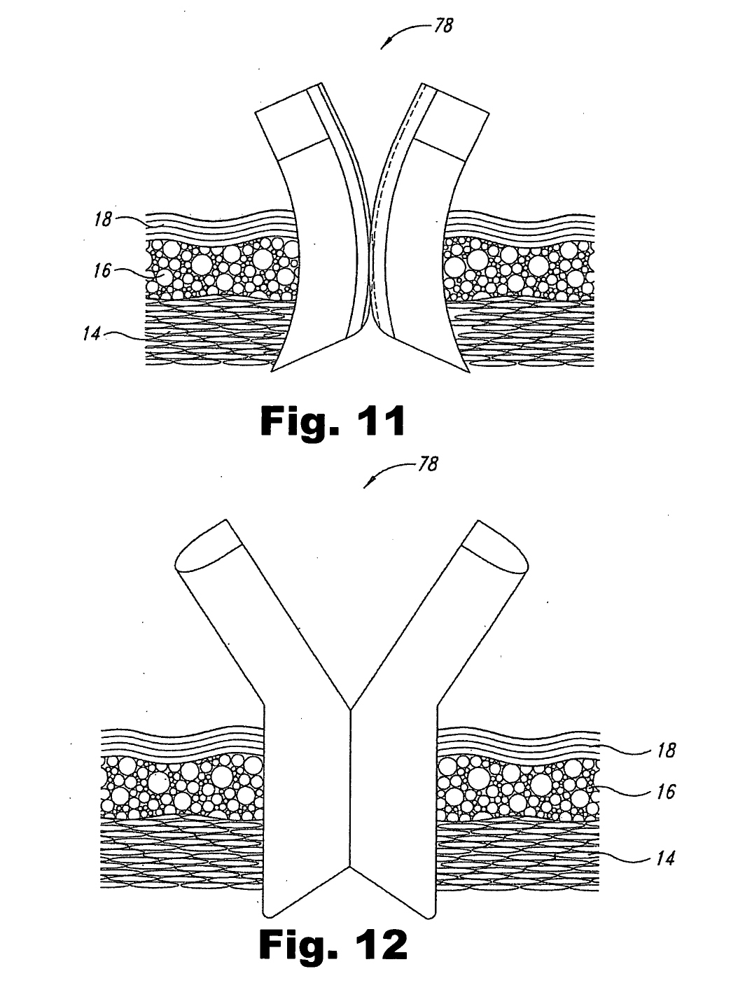

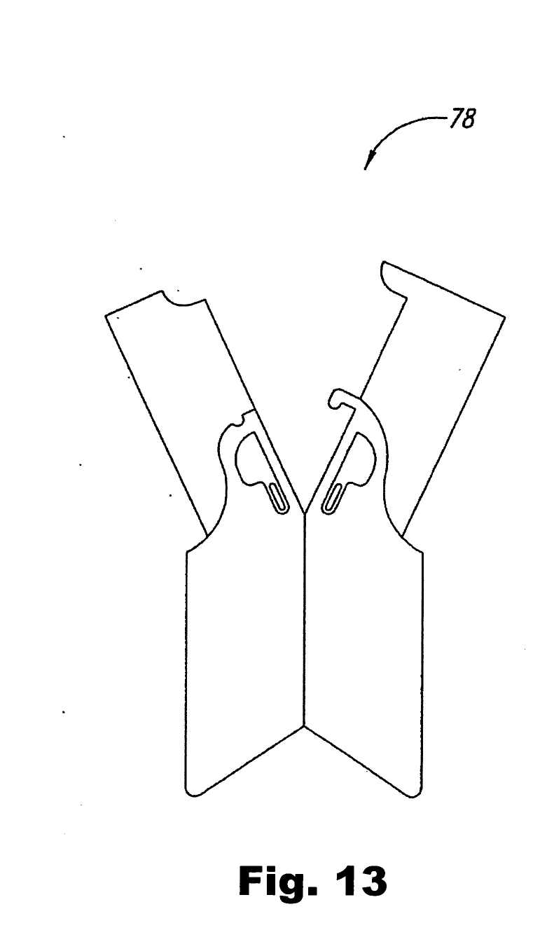

After preparing the opening in the vertebral body (12), securing the screw (48) therein, placing and expanding the blade members (32), the surgeon may elect to insert a minimally invasive surgical (MIS) retractor or MIS port (78) as shown in FIG. 11. The MIS port (78) can be guided over the wire (60) through the interstices (64). FIGS. 12 and 13 show two additional embodiments of the MIS port (78) that may be used with the present exemplary system. Each of the exemplary embodiments of the MIS port (78) are described in detail in a U.S. Provisional Patent Application, filed on Jul. 29, 2005, entitled “Minimally Invasive Surgical Retractor,” having, Provisional Patent Application No. 60/703,606, Express Mail No. EV560405298US and corresponding to Attorney Docket No. 40359-0056.

In conclusion, the present exemplary systems and methods provide a variety of ways to minimize the trauma and damage that may occur to the tissue in the vicinity of a surgical site. Specifically, a tool includes an elongated driver having an expander and a plurality of blade members, each having a proximal portion and a distal portion. According to this exemplary embodiment, the distal portions of the plurality of blades are positioned around and proximate to the expander. The plurality of blades is moveable from a retracted position to an expanded position. When in the retracted position, the distal portions of the plurality of blades are located a first distance from the driver. However, when in the expanded position, the distal portions of the plurality of blades are located further from the driver than when in the retracted position.

Various embodiments of the present assemblies, devices, and systems have been described herein. It should be recognized, however, that these embodiments are merely illustrative of the principles of the present assemblies, devices, and systems. Numerous modifications and adaptations thereof will be apparent to those skilled in the art without departing from the spirit and scope of the present assemblies, devices, and systems.

The various embodiments described above can be combined to provide further embodiments. All of the above U.S. patents, patent applications and publications referred to in this specification, are incorporated herein by reference, in their entirety. Aspects of the invention can be modified, if necessary, to employ devices, features, and concepts of the various patents, applications and publications to provide yet further embodiments of the invention.

The preceding description has been presented only to illustrate and describe exemplary embodiments of the present system and method. It is not intended to be exhaustive or to limit the system and method to any precise form disclosed. Many modifications and variations are possible in light of the above teaching. It is intended that the scope of the system and method be defined by the following claims.

What is claimed is:

1. A tool comprising:
an elongated driver including an expander; and
a plurality of blade members each having a proximal portion and a distal portion, said distal portions of said plurality of blade members being positioned around and proximate to said expander;
wherein said plurality of blade members are moveable from a first retracted position wherein said distal portions of said plurality of blade members are located a first distance from said driver to an expanded position wherein said distal portions of said plurality of blade members are located a second further distance from said driver.

2. The tool of claim 1, wherein said expander comprises:
a campana profiled protrusion; and
a coupling member protruding from said campana profiled protrusion.

3. The tool of claim 2, wherein said coupling member is configured to removably engage a screw.

4. The tool of claim 1, wherein said elongated driver further comprises a handle.

5. The tool of claim 1, further comprising a flexible membrane coupled to said plurality of blade members to form a substantially continuous support member.

6. The tool of claim 1, wherein each of said plurality of blade members further comprise:
an outer surface; and
an inner surface;
wherein said inner surface includes a cam surface configured to interface with said expander.

7. The tool of claim 6, wherein said cam surface comprises an inclined surface formed on an inner surface of at least one of said blade members.

8. The tool of claim 1, further comprising a sleeve closely received by said blade members, wherein said sleeve is configured to retain said blade members proximal to said driver.

9. A percutaneous surgical tool comprising:
an elongated driver including a handle on a first end and an expander having a campana profiled member and a coupling member configured to removably engage a screw protruding from said campana profiled member on a second end;
a plurality of blade members each having a proximal portion, a distal portion, an outer surface, and an inner surface;
wherein said distal portions of said plurality of blade members are positioned around and proximate to said expander;
wherein said inner surface of said blade members includes a cam surface configured to interface with said expander; and
wherein said plurality of blade members are moveable from a first retracted position wherein said distal portions of said plurality of blade members are located a first distance from said driver to an expanded position wherein said distal portions of said plurality of blade members are located a second further distance from said driver.

10. The percutaneous surgical tool of claim 9, further comprising a flexible membrane coupled to said plurality of blade members to form a substantially continuous support member.

11. The percutaneous surgical tool of claim 9, wherein said cam surface comprises an inclined surface formed on an inner surface of at least one of said blade members.

12. The percutaneous surgical tool of claim 9, further comprising a sleeve closely received by said blade members, wherein said sleeve is configured to retain said blade members proximal to said driver.

13. A method for percutaneously preparing a bone to receive a screw, the method comprising:
inserting a wire through human tissue;
guiding said wire through interstices present in said tissue until said wire contacts a portion of said bone;
guiding a tool over said wire;
forming a passage in said bone; and
forming internal screw threads in said passage.

14. The method of claim 13, wherein guiding said wire further comprises viewing said wire in real time as said wire is guided through said tissue using x-ray imaging techniques.

15. The method of claim 14, wherein viewing said wire further comprises imaging said wire using x-ray imaging techniques.

16. The method of claim 14, wherein viewing said wire further comprises imaging said wire using fluoroscopic imaging techniques.

17. The method of claim 13, wherein guiding said wire through interstices present in the tissue comprises:
maneuvering the wire through the tissue without puncturing the tissue with the wire.

18. The method of claim 17, wherein maneuvering said wire through said tissue comprises maneuvering said wire through paraspinous tissue encasing a vertebrae.

19. The method of claim 13, wherein forming said passage in said bone comprises drilling said passage with a drill.

20. A percutaneous method for inserting a screw into a bone, comprising:
inserting a driver coupled to said screw through tissue present around said bone;
turning said screw into said bone with said driver; and
inserting a cannula through said tissue.

21. The method of claim 20, further comprising releasing said screw from said driver after said screw is adequately coupled to said bone.

22. The method of claim 20, further comprising removing said driver.

23. The method of claim 20, further comprising aligning an opening in said cannula with said screw.
